(19) United States
(12) Patent Application Publication
(10) Pub. No.: US 2002/0065432 A1
(43) Pub. Date: May 30, 2002

(54) Method for preparing carbonyl dichloride from chlorine and carbon monoxide

(76) Inventors: Heiner Eckert, Munchen (DE); Bernhard Gruber, Moosburg (DE); Johann Auerweck, Worth (DE)

Correspondence Address: Gary M. Nath, NATH & ASSOCIATES PLLC, 6th Floor, 1030 15th Street, N.W., Washington, DC, 20005, US

(21) Appl. No.: 09/970,894

(22) Filed: Oct. 5, 2001

Related U.S. Application Data
(63) Continuation-in-part of application No. PCT/EP00/03323, filed on Apr. 13, 2000.

(30) Foreign Application Priority Data
Apr. 14, 1999 (DE) 199 16 856.3
Oct. 26, 2000 (WO) 00/63116

(51) Int. Cl. C07C 51/58; C07C 69/96

(52) U.S. Cl. 562/847

(57) ABSTRACT

Industrial methods for producing carbonyl dichloride consist of a two step process with reactors that are connected in series, an exact fine tuning of the reaction parameters and with a high degree of consistency with regard to reaction conditions. This results in a reaction being inefficiently carried out in instances of intermittent operation and decentralization By altering the catalyst, ever higher operation temperatures are required thus having an adverse effect on the quality of the product. Bringing chlorine into contact with activated carbon can induce the formation of tetrachloromethane. Other catalysts should enable the synthesis of carbonyl dichloride in a manner which is scalable, technically simple and, to the greatest possible extent, free of by-products. The invention relates to a method for carrying out the scalable production of carbonyl dichloride from chlorine and carbon dioxide by reacting chlorine and carbon dioxide on a catalyst selected from the row of metal halogenides, whereby the reaction parameters that include pressure and temperature can be selected within a wide range, and the production of by-products is either diminished or prevented. The inventive method makes it possible to synthesize high quality carbonyl dichloride from chlorine and carbon monoxide in various orders of magnitude while using variable reaction parameters.

The preparation of carbonyl dichloride, one of the most diverse and most frequently produced synthetic chemicals (about 5 mn. tons worldwide in 1996; Henri Ulrich, “Chemistry and Technology of Isocyanates”, John Wiley, New York, 1996; Kirk- Othmer, “Encyclopedia of Chemical Technology”, 4^{th} ed., vol. 18, John Wiley, New York, 1996; Ullmann's Encyclopedia of Industrial Chemistry, 5^{th} ed., vol. A19, Verlag Chemie, Weinheim, 1991), from chlorine and carbon monoxide on a catalyst is well known and an industrial-scale process described in detail in the Bayer AG patent DE 3 327 274 (applied for on Jul. 28, 1983). In this process, activated carbon is used as a catalyst and the heat of reaction drawn off by special cooling systems. In addition, the following points characterise the previous major industry processes:

The processes rely on the exact fine tuning of the reaction parameters and a high degree of consistency of the reaction conditions. Any variations result in considerable losses in yield of carbonyl chloride. The process comprises a long start-up phase and cannot be interrupted without significant time losses.

The methods consist of a two-stage process with reactors placed in series. This makes reactions in the batch mode inefficient.

Due to the fact that certain reaction conditions must be observed, every plant must be defined with regard to type and, particularly, magnitude. As a result, scalability, i.e. direct transfer of magnitudes, is not possible.

The catalyst ages with extended operation and, in the course of time, requires ever increasing operating temperatures which, in turn, has an adverse effect on the composition of the product gas: the reverse reaction of carbonyl dichloride to yield chlorine and carbon monoxide increases significantly, which requires time consuming gas scrubbing.

The catalyst of activated carbon itself triggers side reactions. The comparatively long contact time of chlorine on large surfaces of activated carbon at elevated temperatures induces formation of tetrachloromethane which is a toxic by-product severely affecting the polycarbonate production and, in addition, is harmful to the environment. Concentrations of about 400 ppm of tetrachloromethane and above are typical.

Especially the decrease of the tetrachloromethane content has induced intensive research efforts. This resulted in applications for protective rights (e.g. WO 98/00364, applied for on Jun. 28, 1996) describing a decrease of the tetrachloromethane content in the carbonyl dichloride to below 100 ppm. This is achieved by modifying the activated carbon catalyst by partially loading it with metals in the per mil range.

Another process of Bayer AG (DE 19 543 678, application dated Nov. 23, 1995) describes an electrochemical process for preparing carbonyl dichloride from hydrogen chloride and carbon monoxide with the concomitant production of hydrogen on the one hand and, with exposure to oxygen, the concomitant production of water on the other.

A new process (DE 197 40 577, application of Sep. 15, 1997) comprises the formation of carbonyl dichloride from bis(trichloromethyl) carbonate.

Formation from chlorine and carbon monoxide is still important for the industrial production of carbonyl dichloride. It is the object to design the process in a manner permitting a one-stage process and variation of the reaction parameters pressure and temperature over a wide range so that a maximum of flexibility in controlling the process may be achieved and interfering by-products such as tetrachloromethane are minimised.

This object is achieved by reacting chlorine and carbon monoxide on a catalyst selected from the series of metal halides.

It is an essential advantage of the invention that quantitatively pure carbonyl dichloride is produced from chlorine and carbon monoxide on metal halides without activated carbon even at room temperature and significantly below. This catalyst may be present in pure form or applied on a support. This process may be conducted both continuously and batchwise. Pressure during the reaction may range from normal pressure to 100 bar; a slight excess pressure of 0.2 to 15 bar has turned out to be advantageous.

Unexpectedly, this process works at temperatures of −30° C. to 300° C., 0° C. to 100° C. being preferred. Suitable catalysts are metal halides, preferably of metals from the 3^{rd} main group of the Periodic Table of Elements. Aluminium chloride and the gallium chlorides Ga(II) and Ga(III) chloride are particularly well suited. The reactivity of the latter exceeds that of aluminium chloride by several orders of magnitude. Mixed halides of metal alloy components are also suitable catalysts.

A special advantage of the process lies in the very absence of activated carbon as catalyst, because this means that no tetrachloromethane or other chlorinated compounds that might adversely affect further reactions and/or be severely harmful to the environment can be formed from the reaction of chlorine with activated carbon.

Another advantage of the process is its scalability. It is possible to translate an embodiment into practically any order to magnitude.

In a special embodiment, the process permits automatic regeneration of the metal halide catalyst by keeping it consistently active through continued sublimation during the reaction.

In the following, the process is illustrated in greater detail by way of example.

### EXAMPLES 1 - 7

1 mmol of catalyst is fed into an autoclave having a volume of 100 ml and equipped with a magnetic stirrer and chlorine and carbon monoxide are added under pressure of 6 bar and 12 bar, respectively. The reaction is allowed to proceed with stirring at the pertinent heating temperature until the pressure drop is completed and the pressure after cooling to room temperature is about 6 bar. After that the autoclave is cooled to −20° C. and relaxed gently. The autoclave is then weighed (in the cooled condition), subsquently heated to 100° C., the product recondensed in a cold trap and the autoclave weighed back. The product yield is determined from the difference in weight; from the condensate, the product is identified as pure carbonyl dichloride (GC).

TABLE 1

| | | Heating | | Carbonyl |
|---|---|---|---|---|
| | | temperature | Reaction | dichloride |
| Example | Catalyst | (° C.) | time | yield (%) |
| 1 | AlCl₃ | 20 | 60 | 100 |
| | | (RT) | | |
| 2 | AlCl₃ | 55 | 18 | 100 |
| 3 | AlCl₃ | 100 | 1.3 | 96 |
| 4 | AlCl₃ | 180 | 0.5 | 100 |
| 5 | 0,25 mmol | 20 | 2 | 92 |
| | GaCl₃ | (RT) | | |
| 6 | CoCl₂ | 150 | 20 | 85 |
| 7 | PdCl₂ | 100 | 110 | 96 |

### EXAMPLES 8-11

In the above experimental arrangement from examples 1 to 7, chloride and carbon monoxide are added under 6 bar each. At room temperature (about 20° C.), the half-life (at 6 bar) and the reaction time (at 3 bar=const.) are determined on the basis of the pressure drop. Work-up is carried out as described above (examples 1 to 7).

TABLE 2

| | | | | Carbonyl |
|---|---|---|---|---|
| | | Half- | Reaction | dichloride |
| Example | Catalyst | life (h) | time (h) | yield (%) |
| 8 | AlCl₃ | 7 | 26 | 96 |
| 9 | AlCl₃ | — | 45 | 100 |
| 10 | GaCl₃ | 0.17 | 1.5 | 100 |
| 11 | GaCl₂ | 0.2 | 1 | 100 |

### EXAMPLE 12

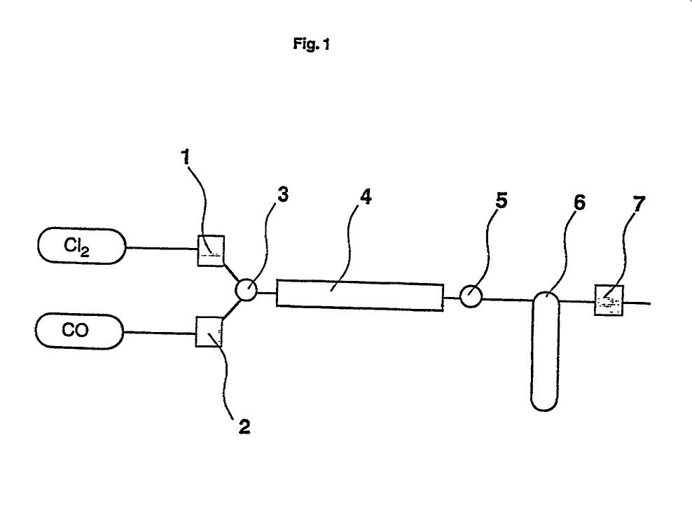

Through a device as shown in FIG. 1, equal parts of (1) chlorine and (2) carbon monoxide in admixture (3) are fed through the metering units into the reaction space (4) consisting of a glass tube (1=350 mm, d=17,5) mm filled with glass wool at the ends and with catalyst (25 g of gallium(III) chloride) in the middle so that an active catalyst stretch of about 100 mm is present. The reaction space (4) is heated to 100° C. by means of infrared heating. The pressure valve (5) adjusted to an excess pressure of 0.3 bar is positioned downstream of the reaction space (4). From said valve, the product stream enters the condensation space (6) where the product is condensed at −20° C. The final part of the device is a bubble counter (7) which is practically free of back-pressure. The chlorine/monoxide gas stream is adjusted through (1) and (2) in such a manner that no gas stream exits through (7). The condensate in (6) is identified as pure carbonyl dichloride (GC).

1. A method for preparing carbonyl dichloride from chlorine and carbon monoxide, characterised in that chlorine and carbon monoxide react on a catalyst selected from the series of metal halides.

2. A method according to claim 1, characterised in that the catalyst is a metal halide of a metal from the 3^{rd} main group of the Periodic Table of the Elements.

3. A method according to claim 1 or 2, characterised in that the catalyst is a metal chloride.

4. A process according to any of the claims 1 to 3, characterised in that the catalyst is aluminium chloride.

5. A process according to claims 1 to 3, characterised in that the catalyst is gallium(II) chloride and/or gallium(III) chloride.

6. A process according to any of the claims 1 to 5, characterised in that the catalyst is a mixed chloride of metal alloy components.

7. A process according to any of the claims 1 to 6, characterised in that the catalyst is applied to a support material which is free of elementary carbon, especially free of activated carbon.

8. A process according to any of the claims 1 to 7, characterised in that the catalytic process of the method takes place in and on materials which do not comprise elementary carbon, especially no activated carbon.

9. A process according to any of the claims 1 to 8, characterised in that the reaction temperature is −30° C. to 300° C., preferably 0° C. to 100 ° C.

10. A process according to any of the claims 1 to 8, characterised in that the pressure during the reaction is the normal pressure (1 bar) to 100 bar, preferably 2 to 15bar.

11. A process according to any of the claims 1 to 10, characterised in that the reaction is continuous.

12. A process according to any of the claims 1 to 11, characterised in that the reaction takes place in a tubular reactor.

13. A process according to any of the claims 1 to 10, characterised in that the reaction is a batch reaction.

14. A process according to any of the claims 1 to 13, characterised in that the reaction takes place in a pressurised vessel.

15. A process according to any of the claims 1 to 14, characterised in that the catalyst is kept consistently active during the reaction by continued sublimation.

16. A process according to any of the claims 1 to 15, characterised in that a maximum of 20 ppm, especially a maximum of 1 ppm of tetrachloromethane is generated during the reaction.

17. A process according to any of the claims 1 to 16, characterised in that the process is a one-stage process.

18. A process according to any of the claims 1 to 17, characterised in that the process is scalable.
